(19) United States
(12) Patent Application Publication
(10) Pub. No.: US 2026/0028564 A1
(43) Pub. Date: Jan. 29, 2026

(54) MICROALGAE CULTIVATION PHOTOBIOREACTOR WITH THE ABILITY TO CLASSIFY THE CULTIVATED ALGAE AND REFINE THE FINAL PRODUCT FOR USE IN ANIMAL AND POULTRY FOOD COMPOUNDS AND HUMAN FOOD ADDITIVES

(71) Applicant: Majid Hajibarati, Tehran (IR)

(72) Inventor: Majid Hajibarati, Tehran (IR)

(21) Appl. No.: 18/786,715

(22) Filed: Jul. 29, 2024

(51) Int. Cl. C12M 1/00; C12M 1/34

(52) U.S. Cl. CPC C12M 21/02; C12M 23/22; C12M 23/40; C12M 29/06; C12M 41/26; C12M 41/34; C12M 41/46; C12M 47/14

(57) ABSTRACT

The present invention relates to a photobioreactor designed for the cultivation of microalgae, specifically the microalga Chlorella, with applications in animal and poultry feed as well as human food additives. This invention falls within the fields of microbiology, food preparation, human and animal nutrition, and agricultural industry practices. The photobioreactor is engineered to enable cost-effective mass production of microalgae by incorporating systems for injecting micronutrients into the culture medium. Additionally, the invention includes a classification system for sorting the produced microalgae and a refinement process for preparing the classified microalgae as a final product suitable for use in various nutritional compounds.

### TECHNICAL FIELD OF INVENTION

The present invention relates generally to the field of microbiology, food preparation, and human and animal nutrition, as well as use in the agricultural industry, and more specifically relates to the cultivation of microalgae and the mass production of microalgae in a cost-effective manner, and in particular to the microalga Chlorella, and also relates to a photobioreactor for cultivating microalgae with the ability to inject micronutrients into the culture medium, and also relates to a system for classifying the produced microalgae, refining the microalgae removed from the classifier for use as a final product.

### PRIOR ARTS

In recent years, there has been extensive research on the development of new healthy foods (nutritional supplements, functional foods, nutraceuticals) derived from microalgal biomass. Microalgae are now available on the market not only in the form of tablets, capsules, and liquids but also in pastas, snacks, candies, beverages, and other food products, either as a dietary supplement or as a natural coloring source. Microalgae are promising living organisms for the sustainable production of food, feed for livestock, poultry, and aquaculture, pure chemicals, and biofuels. Microalgae are also recognized as valuable nutritional and environmental resources in the food and agricultural industries, including protein, mineral, and unsaturated fatty acid rich dietary supplements, food additives in the production of beverages, juices, cheese, cakes, and bread, as well as use as organic fertilizers or as components of composts and organic fertilizers for plant growth and increased crop yields.

Among these, Chlorella species can be cultivated in large quantities, and their dietary supplement products are commercially available worldwide. However, commercial biomass cultivation of Chlorella began only a few years ago. Studies have demonstrated that Chlorella cells contain a variety of nutrients and bioactive compounds that promote human health and prevent specific diseases, suggesting that natural Chlorella-derived compounds could serve as alternatives to synthetic compounds or drugs. The content of these natural compounds in Chlorella varies significantly depending on the cultivation conditions and the specific Chlorella species.

Chlorella is a valuable food source, renowned for its high protein content and essential nutrients. When dried, it comprises approximately 45% protein, 20% fat, 20% carbohydrates, 5% fiber, and 10% minerals and vitamins. Presently, it is cultivated using mass production techniques in large, circular artificial ponds. Chlorella is often marketed as a “superfood” and can be incorporated into various liquid-based nutritional cocktails.

Growth conditions such as temperature, nutrient composition, and light availability significantly influence the levels of biomass, macronutrients, micronutrients, and other valuable bioactive compounds, including antioxidants, in Chlorella cells. The optimal water temperature for Chlorella growth typically ranges between 20 and 30 degrees Celsius. While higher temperatures can accelerate the growth rate, excessively high temperatures can damage the algae.

Chlorella is capable of thriving in a variety of environments, including aquatic settings such as ponds and reservoirs, as well as terrestrial environments within agricultural settings. For optimal growth, Chlorella requires an array of nutrients, including nitrogen, phosphorus, potassium, iron, and other essential minerals. The addition of specialized fertilizers to the growth medium can significantly enhance the growth rate and development of Chlorella. Moreover, atmospheric carbon dioxide is crucial for cultivating Chlorella algae, as CO₂ serves as a vital carbon source for photosynthesis and food production.

As a result, numerous studies have been conducted and various patents have been registered in the field of microalgae cultivation and utilization, particularly focusing on Chlorella. Some notable examples of these inventions are as follows:

A Chinese invention with publication No. CN109679840A, filed on 16 Jan. 2019, titled “Microalgae Batch Conservation Culture Apparatus and Method by the Device Batch Culture Microalgae,” describes a microalgae batch conservation culture apparatus. This apparatus includes a sequentially arranged lighting device, radiator, temperature regulating device, and control circuit, all connected to the control circuit. The lighting device features a square hollow structure with a control panel and several LED light strings arranged in parallel beneath it. The radiator, also square and hollow, has uniformly distributed radiator fans on two sides. The temperature regulating device comprises an aluminum constant temperature spill orifice plate, a semiconductor refrigerating component, an aluminum constant temperature plate, and a heating sheet, arranged in parallel from top to bottom. The apparatus uses LED light strings to provide uniform illumination and controllable precise microdisk electrode temperature, with the temperature regulating device ensuring effective batch conservation of microalgae. The types of microalgae include freshwater algae such as limnetic Chlorella, Scenedesmus quadricauda, or Scenedesmus obliquus, and the cultivation temperature is maintained at 26° C.

Another Chinese invention, publication No. CN102224236A, filed on 30 Oct. 2009, titled “A Photobioreactor,” describes a photobioreactor comprising a base, a supportive frame extending upwardly from the base, and a plurality of trays for culturing phototrophic microorganisms. These trays are arranged vertically from the uppermost tray to the bottommost tray, supported coaxially on the supportive frame, and spaced apart at predetermined gaps to optimize exposure to a light source. A protective member is mounted on top of the uppermost tray. The aim of this invention is to provide a bioreactor that can produce a large amount of microalgae in a limited soil space using minimal energy, thereby reducing production costs compared to encapsulated types requiring high concentrations of CO₂ or oxygen removers. Another objective is to optimize light harvesting (preferably daylight) for microalgae cultivation. To achieve higher output, the operating parameters of the bioreactor must be maintained within a narrow range. Therefore, this invention includes devices for detecting or monitoring the physicochemical parameters of microalgae cultivation in the trays.

Furthermore, Chinese patent No. CN107384802A, granted on 7 Sep. 2021, titled ‘A Method for Promoting Microalgae Grease Accumulation and Maintaining High-Biomass Microalgae,’ discloses a technique for enhancing grease accumulation in microalgae while maintaining high biomass. Specifically, microalgae, after enrichment culture, are placed in a nitrogen-deficient culture medium supplemented with plant hormones for cultivation. The invention introduces the use of plant hormone heteroauxin in nitrogen-deficient culture media to stimulate grease accumulation in microalgae while preserving their biomass. Tests have shown that this method leads to increased fat content in Chlorella under nitrogen stress conditions when plant hormones are added, making it a promising approach for producing microalgae-derived grease.

A Korean invention with Patent No. KR20190087938A, granted on 1 Nov. 2019 and titled “Production Method of High Unsaturated Fatty Acid Using Marine Microalgae,” describes a process for producing highly unsaturated fatty acids using marine microalgae. This invention offers a simpler method compared to the conventional two-stage processes that involve different media and nutrient inhibition techniques. Instead, it employs a single medium and uses specific light wavelengths for oil accumulation, bypassing the need for nutrient inhibition. The LED light wavelength in the first section is blue (460-470 nm), while in the second section, it can be either green (515-525 nm) or red (655-665 nm). The culture periods are 11 days for the one-section phase, three days for the two-section phase, and six days for the three-section phase. The low temperature for all three sections ranges between 0° C. and 20° C. The unsaturated fatty acids produced in these stages may include palmitoleic acid, oleic acid, linoleic acid, linolenic acid, EPA, or DHA.

A U.S. invention, patent No. U.S. Pat. No. 3,142,135A, granted on 28 Jul. 1964 and titled “Production of Carotenoids by the Cultivation of Algae,” details an improved method for producing beta-carotene and xanthophyll through the cultivation of grass green algae from the botanical division Chlorophyta. This method involves cultivating algae in an aqueous organic medium under conditions optimized for maximum carotenoid production. The process entails growing a green alga of the division Chlorophyta in an aqueous organic nutrient medium containing a fermentable carbohydrate, a proteinaceous material, and more than about 0.05 percent by weight of an inorganic nitrate with a cation from the group consisting of metals and ammonium. The process is conducted under heterotrophic conditions, meaning photosynthesis is not involved, and a carbon dioxide source is unnecessary. However, aeration and agitation are preferred to accompany the process.

Another US invention, published as No. US20110256268A1 on 14 Apr. 2011, titled “Oleaginous Yeast Food Compositions,” introduces novel oleaginous yeast biomass, yeast oil, and food compositions containing oleaginous yeast biomass, whole oleaginous yeast cells, and/or yeast oil combined with edible ingredients. The invention focuses on a food ingredient composition that includes a dried egg product and oleaginous yeast flour, which is a homogenate of oleaginous yeast biomass predominantly or completely lysed into powder form, containing at least 25%, 50%, or 75% triglyceride oil by dry weight. The method involves determining the amount of non-oleaginous yeast oil, non-oleaginous yeast fat, or egg in a conventional food product and then replacing or supplementing it with a specified amount of oleaginous yeast biomass.

A Japanese invention, patent No. JP5865894B2, granted on 17 Feb. 2016 and titled “High Lipid Microalgae Powder Food Composition,” describes a homogenate of microalgal biomass. This composition comprises: (a) algae powder, predominantly lysed cells containing more than 20% triglyceride oil by dry weight, or entirely lysed cells; (b) at least one additional edible ingredient, and optionally other ingredients; and (c) gas within the continuous phase of the algae powder and additional edible ingredients. The gas constitutes a discontinuous phase, making up 1% to 50% of the food volume. In some examples, the gas volume is about 10% to 60%. The algal powder or biomass contains 20% to 70% triglyceride oil by dry weight, with 60% to 75% of the triglyceride oil being 18:1 lipid in glycerolipid form.

A US invention, publication No. US20100303990A1, filed on 8 Jan. 2010, titled “High Protein and High Fiber Algal Food Materials,” provides microalgal biomass high in protein and fiber, produced through heterotrophic fermentation. These materials are valuable for manufacturing meat substitutes, meat enhancers, and other food products that benefit from the addition of digestible protein and dietary fiber. The structural properties of foods, such as texture and water retention, are enhanced using these materials. The high protein and fiber food materials of this invention can be manufactured from both edible and inedible heterotrophic fermentation feedstocks, including corn starch, sugar cane, glycerol, and depolymerized cellulose. Generally, this invention relates to a microalgal flour, which is a homogenate of microalgal biomass containing predominantly or completely lysed cells in the form of a powder. The algal biomass comprises at least 40% protein by dry weight and less than 20% triglyceride oil by dry weight and is derived from algae heterotrophically cultured and processed under good manufacturing practice (GMP) conditions.

Another US invention, publication No. US20100297295A1, filed on 8 Jan. 2010, titled “Microalgae-Based Beverages,” describes microalgae-containing gluten-reduced and gluten-free finished food compositions, as well as microalgae-containing food ingredients for large-scale production of gluten-reduced and gluten-free foods. The foods and ingredients of this invention not only reduce or eliminate gluten but also enhance health benefits by replacing less healthy oils and fats with primarily monounsaturated algal oils. The invention also discloses methods for reducing food allergies and symptoms of diseases such as Celiac-Sprue, addressing the increasing sensitivity to gluten-containing products. The food product is formed by combining microalgal biomass, comprising at least 16% triglyceride oil by dry weight, with at least one other gluten-free flour or gluten-free grain product.

Another US invention, with publication No. US20100303989A1, filed on 8 Jan. 2010, titled “Microalgal Flour,” pertains to the development of egg-based food products incorporating various raw materials derived from microalgae in different forms. These forms include high levels of monounsaturated oil, dietary fiber, carotenoids, and digestible crude protein. The invention provides methods and compositions to enhance food stability at elevated temperatures during extended storage periods in hydrated egg products. The microalgae-derived materials are presented as dry or hydrated homogenates produced heterotrophically from different genera, species, and strains of microalgae. This invention reduces the weight/weight levels of saturated fats and cholesterol in egg products while increasing dietary fiber content. Additionally, it offers unique combinations of egg whites and microalgae to manufacture very low cholesterol egg products. In some embodiments, the textural characteristics of powdered eggs are modified to resemble those of liquid eggs by incorporating dietary fiber and other moisture-retaining properties of microalgal biomass.

A Chinese invention with patent No. CN115418320B, granted on 22 Sep. 2023, titled “Chlorella Pyrenoidosa with High Protein Yield, and Culture Method and Application Thereof,” introduces microalgae-containing baked goods with enhanced properties compared to existing products. This invention discloses methods for formulating and manufacturing these foods to achieve reduced fat, lower cholesterol, and increased fiber content. Various embodiments include the elimination or reduction of eggs, butter, animal fat, and saturated oils, substituting them with healthy oil-containing microalgae biomass and oils. The result is the production of foods with lower caloric content than traditional counterparts. The invention provides a food product formed by baking a mixture of microalgal biomass, with a triglyceride oil content of at least 16% by weight, in the form of whole cell flakes, whole cell powder, or a homogenate containing predominantly or completely lysed cells, combined with an edible liquid and at least one other edible ingredient. In some instances, the microalgal biomass is in the form of microalgal flour, which is a homogenate of microalgal biomass containing predominantly or completely lysed cells in powdered form.

Another Chinese invention, Patent No. CN114343021B, granted on 5 Apr. 2024, titled “Application of DHA Algae Oil for Improving Fishy Smell in Edible Oil,” describes a method to enhance edible oil by incorporating DHA algae oil. This edible oil includes a base oil and DHA algae oil, where the DHA algae oil has a carotenoid content exceeding 1.5 mg/kg, an anisidine value of less than 1.0, and a peroxide value of less than 1 meq/kg. The DHA algae oil is derived from microbial oil that undergoes refining processes, including at least two degumming steps and a deodorizing treatment. The specific deodorization process involves introducing steam into the microbial oil under a vacuum of less than 1000 Pa, with the steam at 80-120° C. and deodorization occurring at 140-160° C. The resulting edible oil, enhanced with DHA algae oil, exhibits no noticeable off-flavors and maintains a high DHA retention rate during high-temperature cooking, thereby offering health benefits to consumers.

A Canadian invention, published as No. CA2992860A1 and filed on 24 Jul. 2015, titled “A Protein Rich Food Ingredient from Biomass and Methods of Production,” provides a protein material and food ingredient derived from a sustainable and stable source such as cellular biomass, including algal or microbial biomass. This invention outlines a method for producing a protein composition with high nutritional content and desirable organoleptic properties through a series of steps. The process involves:
1. Pasteurizing the cellular biomass to produce a pasteurized biomass.
2. Exposing the pasteurized biomass to acidic conditions by adjusting its pH to less than 4.5 and maintaining this depressed pH for at least 10 minutes to convert proto-protein into the protein composition.

The acidic conditions specifically involve a pH of less than 4.0, with the pH maintained for at least 20 minutes. The biomass is subjected to these conditions by contacting it with an inorganic acid, such as sulfuric acid or hydrochloric acid. This method effectively yields a protein composition suitable for use as a food ingredient.

A Japanese invention, patent No. JP5828238B2, granted on 2 Dec. 2015 and titled “Apparatus and Method for Culturing Microalgae,” describes a microalgae culture apparatus and method involving one or multiple algal carriers extending vertically. The culture solution is supplied from the upper part of the algal carriers. The apparatus includes a culture solution supply means located below the algae carrier, a culture solution storage tank open at the top, a pump for drawing liquid from the storage tank and delivering it to the upper part of the algal carrier, and a sterilization device for sterilizing various bacteria contained in the culture solution. Additionally, it features a temperature adjusting device to regulate the culture solution temperature and a nutrient addition device to ensure the nutrient concentration reaches appropriate levels based on measurements from a nutrient analyzer. The system also incorporates a protective sheet covering the algal carrier and a carrier rotating device for rotating the algal carrier in a suspended state.

Another Japanese invention, patent No. JP6386727B2, granted on May 9, 2018, titled “Food Composition of Microalgal Biomass,” describes a method for producing microalgal flour as a food ingredient. The process includes: (A) providing microalgal cells containing at least 10% triglyceride oil by dry weight, specifically Chlorella protothecoides; (B) disrupting the cells, reducing the particle size, and creating an aqueous homogenate; and (C) drying the homogenate to produce a microalgal powder with at least 10% triglyceride oil by dry weight. This method also involves separating the cells from the culture medium before disruption. The cell disruption is performed using a pressure disruptor, a pressurized cell disruptor, or a ball mill. Drying is carried out using a flash dryer or a spray dryer. The microalgal cells contain 50% to 70% oil by dry weight. The process may include adding a fluidizing agent before drying. The average particle size of the microalgal powder is less than 10 μm, with an average particle diameter of less than 5 μm. The microalgal powder has a water content of 10 wt % or less, or 5 wt % or less, and 50% to 73.83% of the oil is 18:1 lipid in the form of glycerolipid.

A Japanese invention, patent No. JP7049647B2, granted on 7 Apr. 2022, titled “Algae Growing Method and Algae Culture Equipment,” pertains to a device and method for algae cultivation and removal. The device includes a support where algae attach, placed in a gas phase, and continuously supplied with culture solution from above to promote algae growth. The algae are removed from the support every 2 to 4 hours, with the removal amount ranging from 0.1 to 0.6 (where 1.0 represents the algae amount on the support before removal). The removal process involves using a suction recovery device to extract the algae and culture solution. This method allows for the continuous production of new algae with maximum efficiency, significantly enhancing productivity compared to traditional methods.

The support can be any material that algae can adhere to, typically a sheet-like structure, and may also include three-dimensional objects such as cylinders.

Another Japanese invention, patent No. JP6152933B2, granted on 28 Jun. 2017, titled “Chlorella Culture System and Chlorella Culture Method,” describes a culture system and method for microorganisms, including photosynthetic microorganisms such as microalgae. The microorganism culture system comprises a carrier placed in a gas phase to which microorganisms adhere, an effluent tank that stores a culture solution containing microorganisms that have flowed out of the carrier, and a continuous top surface of the carrier. The culture fluid flows over the surface of the carrier at a rate between 5 mL/h/m² and 1200 mL/h/m², allowing chlorella to grow on the carrier surface where the culture fluid flows naturally. This system is designed to collect chlorella from the culture solution that has naturally flowed into the tank. Additionally, it includes a flow path for supplying a culture solution, from which at least some chlorella has been removed, from the effluent tank to the carrier. The invention preferably features a light irradiation unit for illuminating the carrier. The aerated CO₂ mixed air does not pass through water, enabling normal pressure ventilation. This method allows for the collection of microalgae in a concentrated state by performing continuous culture and forced detachment from the carrier surface as needed.

An article titled “Enhancing Health Benefits through Chlorophylls and Chlorophyll-Rich Agro-Food: A Comprehensive Review,” published on 11 Jul. 2023, discusses the significant role of chlorophyll, the green pigment present in plants, and chlorophyll-rich foods in promoting human health. The review highlights that chlorophylls are essential in photosynthesis and are abundantly present in green fruits and vegetables, which are crucial components of our diet. Although research is limited, existing studies suggest that these photosynthetic pigments and their derivatives possess therapeutic properties. These bioactive molecules exhibit various beneficial effects, including antioxidant, antimutagenic, antigenotoxic, anticancer, and anti-obesogenic activities. The article notes the unfortunate reality that leafy materials and fruit peels often become waste in the food supply chain, contributing to the issue of food waste in modern societies. However, these overlooked materials contain valuable bioactive compounds, including chlorophylls, which offer significant health benefits. Consequently, exploring the potential of these discarded resources, such as utilizing them as functional food ingredients, aligns with the principles of a circular economy and presents exciting opportunities for exploitation.

Another article titled “Extraction of Pigments from Microalgae and Cyanobacteria-A Review on Current Methodologies,” published on 3 Jun. 2021, reviews various methods for extracting pigments from microorganisms, detailing the advantages and disadvantages of each approach. Due to their bioactive potential and natural product attributes, pigments from microalgae and cyanobacteria have garnered significant interest for industrial applications. Typically, these pigments are sold as extracts to circumvent purification costs. The extraction processes rely on cell disruption methodologies and the chemical solubility of the compounds. Techniques used for pigment extraction include sonication, homogenization, high-pressure methods, CO2 supercritical fluid extraction, enzymatic extraction, and emerging methods such as ohmic heating and electric pulse technologies. The primary challenge in pigment bioprocessing arises from the installation and operation costs. Consequently, both fundamental and applied research are necessary to address these constraints and enhance the competitiveness of the microalgae and cyanobacteria industry in the global market. This review discusses the main extraction methodologies, considering the advantages and disadvantages for each pigment type, organism, cost, and final market.

Photobioreactors are extensively utilized in agriculture and cultivation, particularly for algal growth. These reactors supply the essential light energy required for algae to grow and produce food. A typical photobioreactor comprises a culture medium (such as water or another liquid), a photocatalyst (which may be inherent to the algae), and a light source (like a UV lamp or artificial sunlight). When exposed to light, the photocatalysts activate, initiating the chemical reactions necessary for algal growth. The use of photobioreactors in algal cultivation provides numerous benefits, including enhanced control over growth parameters (such as temperature and light), minimized contamination, increased proliferation and growth rates, and improved cultivation and production efficiency. This method is increasingly recognized as a green and sustainable approach for algal production, finding applications across various industries including food, pharmaceuticals, and energy.

### DESCRIPTION OF THE INVENTION

The present invention provides a photobioreactor designed for cultivating microalgae, featuring a helical glass structure capable of injecting essential micronutrients and CO₂ for photosynthesis (FIG. 1). This system incorporates a horizontally positioned helical structure made of glass, optimizing sunlight penetration, a crucial factor for the growth and reproduction of microalgae. The structure measures 10 meters in width and 30 meters in length, although these dimensions can be adjusted. Additionally, the photobioreactor can be housed under a transparent roofing structure to ensure maximum sunlight exposure. The helical design increases the surface area of the culture tanks, enhancing efficiency. In one embodiment, the photobioreactor utilizes microalgae of the Chlorella species.

The glass tubes of this photobioreactor are interconnected at the corners using elbows, allowing the tubes to make multiple turns within the structure before reaching the center. This helical configuration can be replicated numerous times based on the production site's capacity, with two, three, five, or more helical structures positioned alongside each other. The output of each spiral serves as the input for the next (FIG. 2), enabling the construction of up to 1 or 2 kilometers of glass tubing. Using glass as the primary material for the structure facilitates the observation and monitoring of microalgal growth. Additionally, the helical configuration optimizes space and efficiently utilizes sunlight due to the increased surface area of the culture tubes.

The spiral tubes of this invention's helical system are filled with a fluid such as water to create an optimal growth medium for microalgae. To enhance the culture medium, chambers containing macro and micronutrients, including nitrogen, phosphorus, potassium, zinc, copper, and iron, are embedded along one side of the helical structure (see FIGS. 4, 5, and 6, No. 104). These chambers are a series of interconnected containers, each holding a specific nutrient solution. The nutrients are dissolved in water to facilitate their uptake by the microalgae. Pumps inject these nutrient solutions into the helical tubes, ensuring even distribution throughout the structure. The system employs a dosing pump (FIG. 5, No. 103) located at the base of each reservoir, which connects to a collector pipe (FIG. 5, No. 102). This pipe links to a main pump (FIG. 5, No. 101) that delivers the micronutrient solution into the first loop of the reactor, thus initiating the nutrient distribution process.

Additionally, to monitor micronutrient concentration levels in the culture medium, the photobioreactor incorporates several embedded sensors. These sensors continuously measure the concentrations of micronutrients and transmit the data to a central computer. Upon detecting a decrease in any micronutrient, the system sends a command to the dosing pumps associated with the corresponding micronutrient chamber. These pumps then inject a specified amount of the solution, with a predetermined molarity, into the reactor to maintain optimal conditions.

On the opposite side of the helical structure, an oxidation column is situated (FIG. 3) to produce the carbon dioxide gas required for microalgal photosynthesis. The oxidation column is designed with multiple layers and is connected to municipal liquefied petroleum gas (FIG. 3), which typically comprises natural gases like methane, along with sulfur gases and other impurities and byproducts, including organic compounds. A filter is installed at the gas inlet to partially prevent the entry of these impurities, thereby providing a higher quality gas source to the system. During oxidation, methane is converted into carbon dioxide and water. The gas emitted from the oxidation column enters an air washer (FIG. 7). Since the exhaust gas from the oxidation process is hot, water is sprayed onto it in the air washer to cool the gas. Given the low solubility of CO₂ in water (1.45 g/L), only a small portion dissolves in the water, with the remainder entering the helical structure. Additionally, harmful byproducts generated in the oxidation column, such as SO₂ and H₂S, which could damage the microalgae, are washed away by the water in the air washer and subsequently removed from the system.

The CO₂ gas exiting the air washer is injected into the helical section to supply the required CO₂ for the microalgal culture medium. Additionally, nozzles embedded in the photobioreactor introduce the generated gas into the reactor as fine bubbles under pressure. Due to the high dilution of the gases, they diffuse extensively throughout the helical system.

In another aspect of this invention, a pump is integrated into the reactor to facilitate the gentle circulation of the photobioreactor's contents. It is crucial to maintain the culture medium temperature within an optimal range for microalgal growth, typically between 20 and 25 degrees Celsius, depending on the microalgae species. Furthermore, the pH of the culture medium should be adjusted to fall between 7 and 9, which is ideal for microalgal cultivation. To monitor pH variations within the reactor, two or more pH sensors are provided to measure changes in the culture medium solution.

After a specified period, typically 1 to 3 weeks, when sufficient proliferation has occurred and the maturation phase of Chlorella microalgae is complete, the algae are ready for harvesting. The material within the reactor is then pumped (FIG. 8, No. 202) into the classifier section. This section consists of a cylinder (FIG. 8, No. 201) with a centrally positioned membrane (FIG. 9, No. 306) that divides the cylinder into two longitudinal sections.

A mixture of water and cultivated microalgae is pumped (FIG. 8, item 203) into the first section (FIG. 9, section 304) of the cylinder. The pressure differential between the two sections, created by the pump and a solenoid valve located beneath the cylinder, allows water to pass through the membrane while retaining the microalgae in the first section. This setup ensures that the necessary pressure for water to permeate the membrane is maintained.

The membrane is specifically designed to selectively permit water molecules to pass through while blocking microalgae and other larger particles, ensuring precise separation of water from microalgae. Once water passes through the membrane, it permeates into the second section of the cylinder (FIG. 9, item 302), leaving the microalgae in the first section.

A solenoid valve (FIG. 9, item 303) adjacent to the second section is used to briefly reverse the water pressure when opened. This reverse flow of water back through the membrane towards the first section effectively cleans the membrane by dislodging trapped particles from its pores, thus performing a backwash that enhances the membrane's efficiency and extends its lifespan.

The water separated from the microalgae is routed back to the reactor and culture medium via a one-way solenoid valve and a pipe (FIG. 9, No. 301) located beneath the second section of the cylinder. Before re-entering the reactor, this water is filtered to remove any impurities, ensuring the purity and quality of the culture medium.

The microalgae accumulated in the first section of the cylinder are transferred to the refinery section via a solenoid valve and a pipe (FIG. 9, No. 305) located beneath this section. In the refinery section, the microalgae undergo a drying process to prepare them for use as food. This section involves multiple stages, starting with the refinement and purification of the extracted microalgae. This process includes removing contaminants, organic and inorganic residues, and other undesirable particles, potentially through filtration.

Due to the presence of chlorophyll, which imparts an undesirable color, odor, and taste to the final product, this invention utilizes the Pressurized Liquid Extraction (PLE) method to separate chlorophyll, thus improving the color and odor of the final product. In the PLE method, the main goal is to isolate specific compounds such as chlorophyll. The chamber (FIG. 10) containing the sample is filled with a solvent, heated to the desired temperature, and pressurized. The high pressure prevents the solvent from boiling by increasing its boiling point. At high temperature and under liquid conditions, the solvent exhibits a high diffusion coefficient, low viscosity, and high solubility. The elevated temperature reduces the stability of the cell wall and enhances the solvent's flux into the cell, resulting in the extraction of chlorophyll from the cell.

By selecting the appropriate solvent and optimizing conditions, chlorophyll extraction can be conducted in a way that minimizes the impact on other valuable compounds such as proteins, lipids, and carbohydrates. The chosen solvent should selectively extract chlorophyll without extracting other valuable compounds or disrupting the cellular structure. In this invention, ethanol is utilized as the solvent due to its non-toxic nature, stability under various temperature and pressure conditions, and excellent solubility for chlorophyll.

Temperature and pressure are critical parameters in this method and should be adjusted to minimize cell damage while maximizing extraction efficiency. Relatively high temperatures (but not excessively high) are typically required to extract chlorophyll from microalgae effectively. Temperatures below 40 degrees Celsius may reduce extraction efficiency, while temperatures above 80 degrees Celsius increase the risk of degrading sensitive compounds and damaging cellular structures. Therefore, the optimal temperature range for pressurized liquid extraction (PLE) is 40-80 degrees Celsius.

Pressure is applied in the PLE process to enhance extraction efficiency. Higher pressure facilitates solvent penetration into cells, improving extraction yield. Pressures below 10 MPa may not provide sufficient optimization for extraction, whereas pressures above 30 MPa can damage cellular structures. Hence, a pressure range of 10-30 MPa is considered suitable.

In this invention, a temperature of 60 degrees Celsius and a pressure of 20 MPa are used for chlorophyll extraction, providing good efficiency without significant cellular damage. The typical extraction time for PLE of chlorophyll from microalgae ranges between 30 to 90 minutes, ensuring adequate extraction within a practical timeframe.

Following the PLE extraction process, the solution containing chlorophyll and solvent is collected. To prepare the solution for the subsequent steps, appropriate filters are used to remove suspended particles. Initially, primary filtration with a paper filter is employed to eliminate larger particles. Following this, the solution undergoes a secondary filtration using a membrane filter with small pores (0.45 microns or smaller) to capture fine suspended particles. To enhance the filtration rate and ensure the quick passage of the solution through the filter, a vacuum is applied, effectively separating the suspended particles from the solution.

The extracted chlorophyll from microalgae can be utilized in various industries, including food, pharmaceuticals, and cosmetics. In the food industry, chlorophyll serves as a beneficial additive, aiding in detoxification, energy enhancement, and immune system support. Its anti-inflammatory and antimicrobial properties make it valuable in pharmaceutical applications, particularly in topical ointments for treating wounds, burns, and eczema. In the cosmetic industry, chlorophyll is incorporated into creams, lotions, and face masks to promote skin health and reduce the appearance of wrinkles due to its antioxidant and anti-inflammatory effects.

After the separation of chlorophyll, the microalgae are processed into a form suitable for their intended application. This may involve drying, collecting into a powder, or grinding to the desired particle size for various uses. The drying process is a crucial step in refining and processing microalgae, transforming them from a liquid state, or from a state achieved through refining and purification processes, into a solid, dry form.

This invention employs infrared fan drying for the drying process. Infrared fan drying utilizes infrared (IR) energy to transfer heat to the wet material. Infrared radiation with specific wavelengths is emitted onto the wet material, causing heat transfer into the wet particles, which results in the evaporation of moisture. The process involves irradiating the wet material with infrared radiation, which is absorbed by the microalgae, heating them and causing the moisture within to evaporate and release as vapor. The vapor is then separated from the material, leaving a combination of dry solids and water vapor. The water vapor is removed from the drying environment (FIG. 11, No. 403) using fans (FIG. 11, No. 402), resulting in dry, moisture-free material.

Following the drying process, the microalgae are processed into a powder to obtain the final product. A grinder (FIG. 11, No. 401), such as a pin mill, is used to convert the dried microalgae into a fine powder. This step is essential to extend the shelf life, preserve the quality, and enhance the storability of the microalgae.

### BRIEF DESCRIPTION OF FIGURES

FIG. 1 provides an overall view of the microalgae cultivation photobioreactor system as described in the invention.

FIG. 2 illustrates a side view of the microalgae cultivation photobioreactor system described in this invention.

FIG. 3 depicts the oxidation column.

FIG. 4 illustrates a rear view of the chambers that house both macro and micronutrients.

FIG. 5 illustrates a front view of the helical photobioreactor along with its associated chambers for macro and micronutrients. The components are labeled as follows:
101: Pump
102: Collector Pipe
103: Dosing Pump
104: Micronutrient Chambers

FIG. 6 illustrates a side view of the chambers that house both macro and micronutrients, along with the dosing pumps.

FIG. 7 illustrates a view of the air washer.

FIG. 8 illustrates the external view of the classifier cylinder, including the following components:
201: Classifier Cylinder
202: Pump
203: Pump injecting the microalgae and water mixture into the initial section of the classifier

FIG. 9 presents an internal view of the classifier cylinder and membrane, which includes the following components:
301: Water return pipe leading back to the system.
302: The second segment of the cylinder.
303: Solenoid valve.
304: The first segment of the cylinder.
305: Pipe for transferring microalgae to the refining unit.
306: Membrane.

FIG. 10 illustrates an exterior view of the PLE chamber.

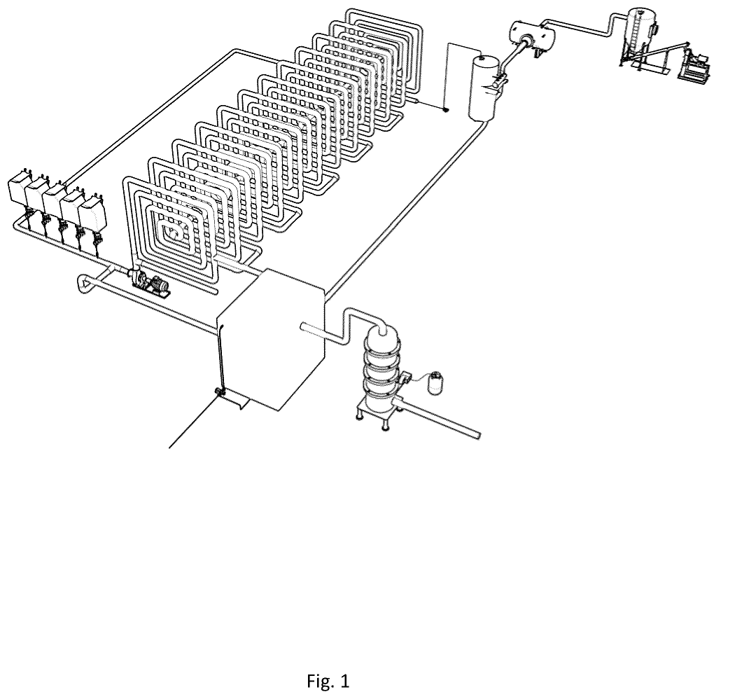

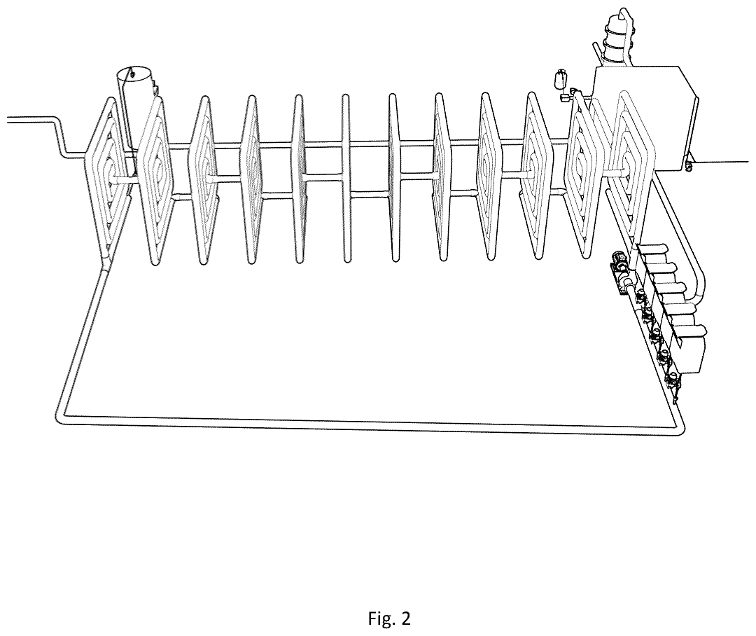

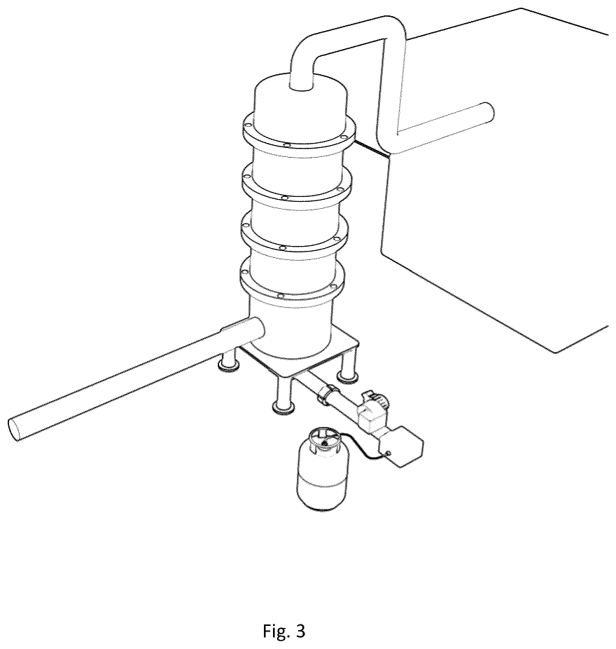

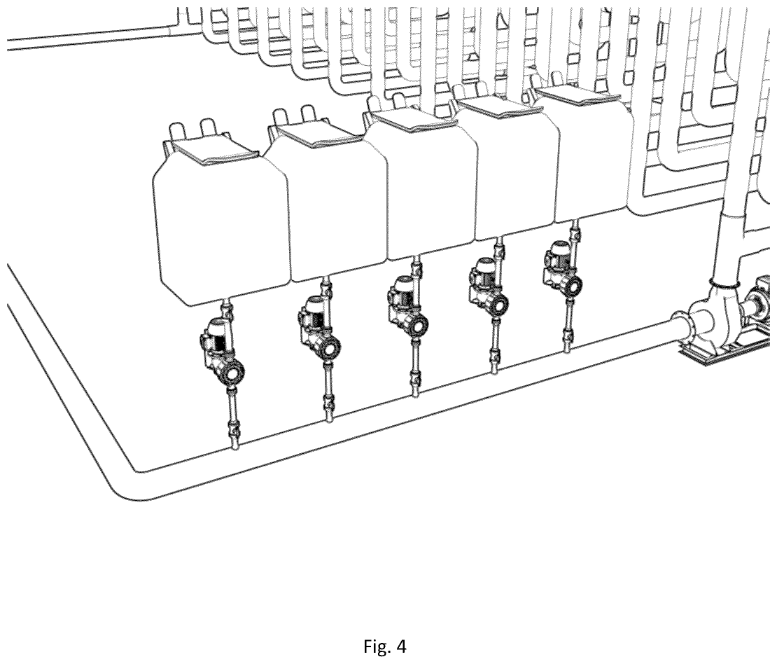

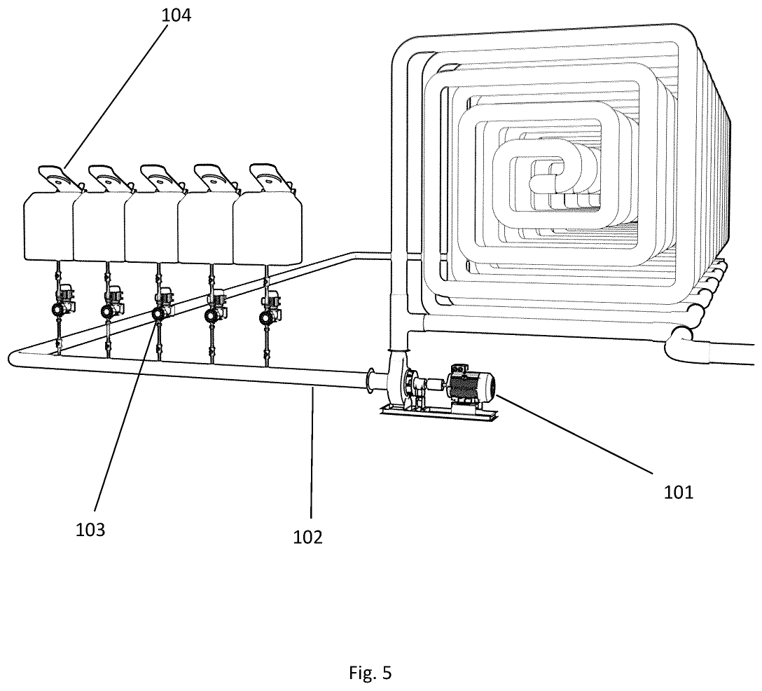

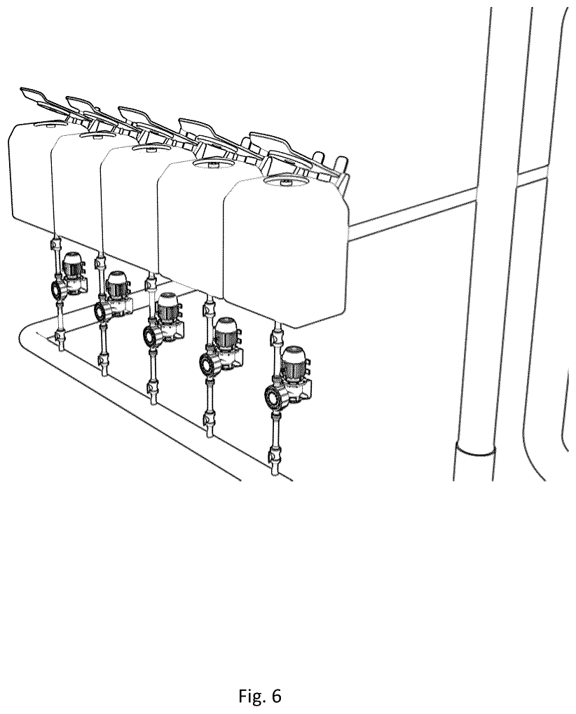

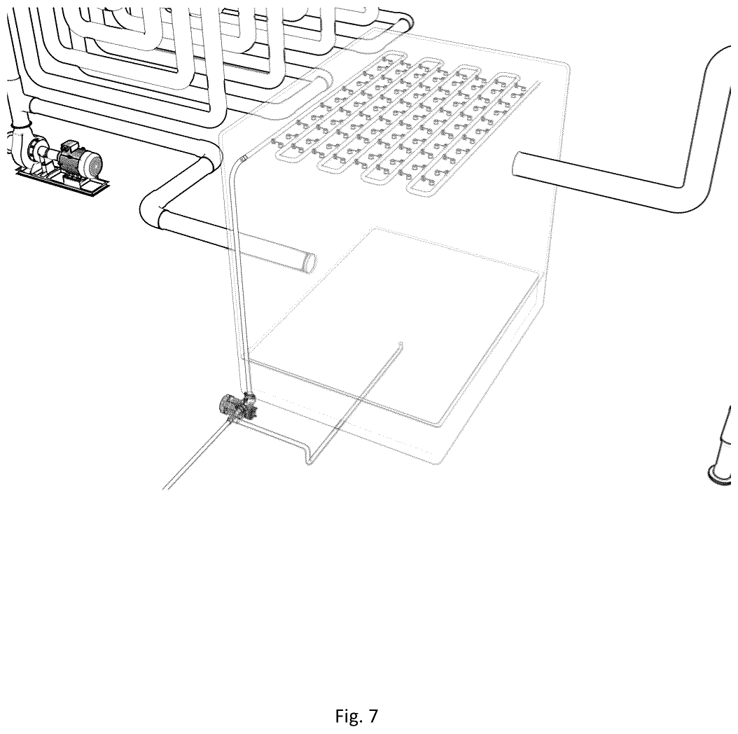

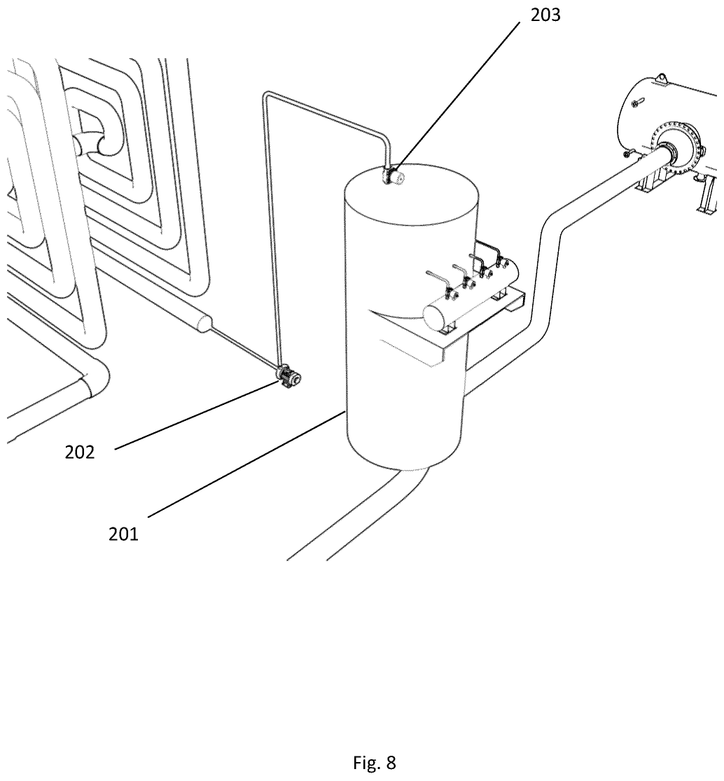

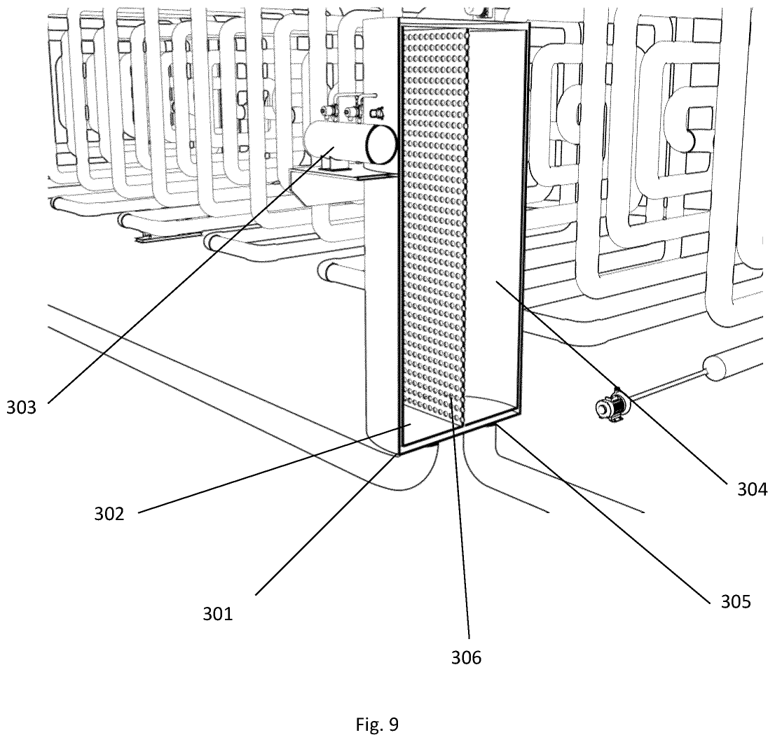

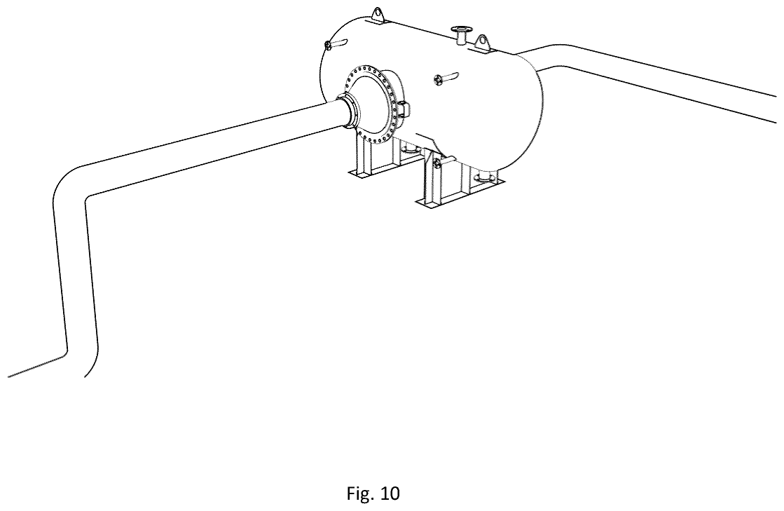

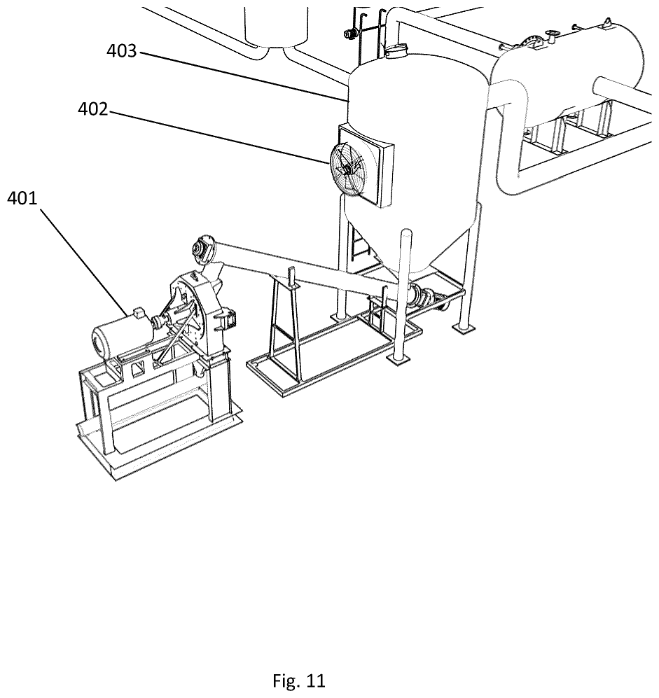

FIG. 11 illustrates the dryer chamber and grinder system, comprising the following components:
401: Pin mill grinder
402: Fan
403: Drying chamber

What is claimed is:

1. The invention of microalgae cultivation photobioreactor with the ability to classify the cultivated algae and refine the final product for use in animal and poultry food compounds and human food additives comprises at least one glass helical photobioreactor, at least some pipe connecting elbow, at least some chamber containing macro and micro nutrients, at least one dosing pump for injecting nutrients into the helical tubes, at least one nutrient collector pipe, at least one nutrient concentration sensor, at least one oxidation column, at least one city gas inlet filter, at least one air washer, at least one carbon dioxide injection nozzle into the photobioreactor, at least one pump for circulating the material in the system, at least one pH sensor, at least one classifier section comprising at least one cylinder and at least one membrane for separating the aqueous phase from the algal phase, at least one water return pump to the photobioreactor environment, at least one refinery section comprising at least one PLE chamber and at least one infrared fan dryer and at least one pin mill.

2. The cultivation system of claim 1, wherein the microalgae photobioreactor is a horizontal helical structure constructed of glass, allowing sunlight—an essential factor for the growth and proliferation of microalgae—to efficiently penetrate and reach the culture medium.

3. The cultivation system of claim 1, wherein, depending on the production site's capacity, the helical structure can be replicated multiple times, with the output of each spiral serving as the input for the subsequent one.

4. The cultivation system of claim 1, wherein chambers containing macro and micronutrients—including nitrogen, phosphorus, potassium, zinc, copper, and iron—are integrated into one side of the helical structure in liquid and water-soluble forms to enhance the absorption rate.

5. The cultivation system of claim 1, wherein a dosing pump is positioned beneath each chamber. All pumps are connected to a collector tube, from which a pump introduces the dissolved micronutrient solution into the first helical section or initial loop of the reactor.

6. The cultivation system of claim 1, wherein multiple sensors are embedded within the photobioreactor to continuously monitor the concentration of micronutrients in the culture medium. These sensors measure micronutrient levels and transmit the data to a central computer.

7. The cultivation system of claim 1, wherein an oxidation column, constructed with multiple layers, is provided to generate the carbon dioxide required for the photosynthesis of microalgae and is connected to a city gas supply.

8. The cultivation system of claim 1, wherein the gas expelled from the oxidation column is directed into an air washer. The air washer cools the exhaust gas, removes impurities, and cleanses the gas before it is released from the system.

9. The cultivation system of claim 1, wherein the CO₂ gas released from the air washer is injected into the system as small bubbles under pressure through nozzles installed in the photobioreactor.

10. The cultivation system of claim 1, wherein a pump is integrated into the reactor to gently circulate the contents of the photobioreactor.

11. The cultivation system of claim 1, wherein the ambient temperature is maintained between 20° C. and 25° C. and the pH is adjusted to a range of 7 to 9, optimizing conditions for the growth of microalgae.

12. The cultivation system of claim 1, wherein, upon completion of the proliferation process, the material within the reactor is transferred to the classifier section. This section comprises a vertical cylinder with a central membrane designed to separate the aqueous phase from the algal phase.

13. The cultivation system of claim 1, wherein the aqueous phase separated in the classifier section is recirculated to the reactor and culture medium by a pump to reduce water wastage.

14. The cultivation system of claim 1, wherein the use of the pressurized liquid extraction (PLE) method to separate chlorophyll effectively reduces the color and odor of the final product.

15. The cultivation system of claim 1, wherein the PLE process is conducted at a temperature of 60 degrees Celsius and a pressure of 20 MPa, utilizing ethanol as the solvent for extracting chlorophyll.

16. The cultivation system of claim 1, wherein paper filtration and a membrane filter with a pore diameter of 0.45 microns are employed to separate microalgae from a solution of ethanol and chlorophyll.

17. The cultivation system of claim 1, wherein infrared (IR) fans are employed to utilize infrared energy for drying microalgae.

18. The cultivation system of claim 1, wherein a pin mill machine is employed to grind dry microalgae into a powdered form.
